(19) United States
(12) Patent Application Publication
(10) Pub. No.: US 2019/0309348 A1
(43) Pub. Date: Oct. 10, 2019

(54) METHOD FOR CHARACTERIZING AFFINITY AGENTS AND RELATED APPARATUS

(71) Applicant: Invoy Holdings, LLC, Aliso Viejo, CA (US)

(72) Inventors: Lubna M. Ahmad, Chandler, AZ (US); Brent Satterfield, Greenwood, SC (US)

(21) Appl. No.: 16/178,058

(22) Filed: Nov. 1, 2018

Related U.S. Application Data
(63) Continuation of application No. 11/804,016, filed on May 16, 2007, now abandoned.

(51) Int. Cl. C12Q 1/6813; C40B 40/08; C40B 30/04; C12Q 1/6844

(52) U.S. Cl. CPC C12Q 1/6813; C40B 40/08; C12Q 2539/115; C12Q 1/6844; C12Q 2563/149; C40B 30/04

(57) ABSTRACT

A method is provided for characterizing analyte-specific affinity agents having affinity for an analyte. The method includes providing a library of candidate affinity agents, providing a surface suitable for adhesion of the library of candidate affinity agents, and exposing the library of candidate affinity agents to the surface to thereby adhere the library of candidate affinity agents to the surface. It also includes providing an analyte, and causing the analyte to contact the surface to thereby expose the library of candidate affinity agents adhered to the surface to the analyte, to cause selected ones of the candidate affinity agents to react with the analyte to form reaction products, and to create an output fluid that comprises the reaction products. In addition, the method includes analyzing the output fluid to characterize the analyte-specific affinity agents associated with the respective reaction products. The candidate affinity agents may take a number of forms, including nucleic acids, peptides, genomers and others. Related apparatus also are provided.

### BACKGROUND OF THE INVENTION

### Field of the Invention

The present invention relates to methods and apparatus for identifying affinity agents, including but not necessarily limited to oligonucleotides, with affinity for an analyte or analytes. Such affinity agents may be useful in a variety of applications, for example, such as in sensor technologies and therapeutics.

### Description of the Related Art

High-affinity agents such as ligands are of great importance in the biotechnology field and in other areas as well. These agents are designed to bind to target molecules, which can be useful in a variety of applications. A high-affinity binding interaction may be useful, for example, for detecting the presence of the target molecule in a sensor or diagnostic application. This type of interaction also can have applications in the therapeutics field.

The currently-preferred method for developing high-affinity oligonucleotide ligands is the “systemic evolution of ligands in exponential enrichment” (“SELEX”) method. This method is limited, however, for example, in that it requires a substantial amount of time for completion, e.g., typical turnaround being around 10-15 weeks. SELEX also requires amplification at the end of each step, which in certain cases presents a disadvantage in requiring an additional step. Additionally, the SELEX method identifies high-affinity ligands by allowing for target-ligand interaction in solution, and accordingly may not be best suited for applications where the ligand is designed for immobilization to a surface (e.g., in a sensor application). Therefore, there is a need for methods that will overcome some or all of the disadvantages of SELEX.

Aptamers in general have been described as ligands identified through SELEX and as non-naturally occurring, randomized oligonucleotides or nucleotide analogs, which undergo non-Watson-Crick binding interactions with another molecule.

Unfortunately, these definitions of aptamers rule out one of the most significant pools of nucleic acids, i.e., genomers.

### SUMMARY OF THE INVENTION

In accordance with one aspect of the invention, a method is provided for characterizing analyte-specific affinity agents having affinity for an analyte. The method comprises providing a library of candidate affinity agents, providing a surface suitable for adhesion of the library of candidate affinity agents, and exposing the library of candidate affinity agents to the surface to thereby adhere the library of candidate affinity agents to the surface. The method further comprises providing an analyte, and causing the analyte to contact the surface to thereby expose the library of candidate affinity agents adhered to the surface to the analyte, to cause selected ones of the candidate affinity agents to react with the analyte to form reaction products, and to create an output fluid that comprises the reaction products. The method still further comprises analyzing the output fluid to characterize the analyte-specific affinity agents associated with the respective reaction products.

An analyte-specific affinity agent or agents having affinity for an analyte that is identified or otherwise characterized according to the aforementioned method constitutes an additional aspect of the invention.

The provision of the library of candidate affinity agents may comprise providing a nucleic acid within the library of candidate affinity agents. The provision of the library of candidate affinity agents also may comprise providing non-naturally occurring nucleic acids within the library of candidate affinity agents. In addition, the provision of the library of candidate affinity agents may comprise providing a peptide within the library of candidate affinity agents. The provision of the library of candidate affinity agents may also comprise providing RNA within the library of candidate affinity agents.

Preferably, the provision of the library of candidate affinity agents comprises providing a genomer within the library of candidate affinity agents. The genomer optionally may comprise at least a portion of a human genome. The human genome may comprise a coding portion of the human genome, it may comprise a non-coding portion of the human genome, or a combination of these. The genomer also may comprise a mouse genome.

The surface may comprise a silica, for example, such as silica beads. The provision of the surface may comprise providing the surface to comprise a spin column.

Preferably, the exposure of the library of candidate affinity agents to the surface comprises adsorbing the library of candidate affinity agents to the surface nonspecifically.

The provision of the analyte may comprise providing the analyte to comprise an analyte solution, a gas phase, or a liquid phase. The provision of the analyte may also comprise providing the analyte to include at least one of proteins, lipids, carbohydrates, and toxins. The provision of the analyte may comprise providing the analyte to include acetone, a volatile organic compound, a small molecule, and/or at least one constituent of human breath. The provision of the analyte also may comprise providing the analyte to comprise a target for a sensor application or for a therapeutic application.

The analysis of the output fluid may comprise using differential binding, comparing a library-silica affinity to a library-analyte affinity, separating the analyte-specific affinity agents from the analyte, sequencing the analyte-specific affinity agents, isolating the analyte-specific affinity agents, identifying the analyte-specific affinity agents, or a combination of these.

The method for characterizing analyte-specific affinity agents aforedescribed may further comprise separating the analyte-specific affinity agents from the analyte, providing a second surface suitable for adhesion of the analyte-specific affinity agents, and exposing the analyte-specific affinity agents to the second surface to thereby adhere the analyte-specific affinity agents to the second surface. This method further comprises causing the analyte to contact the second surface to thereby expose the analyte-specific affinity agents adhered to the second surface to the analyte, to cause selected ones of the analyte-specific affinity agents to react with the analyte to form second reaction products, and to create a second output fluid that comprises the second reaction products. The method further comprises analyzing the second output fluid to characterize enhanced analyte-specific affinity agents associated with the respective reaction products.

In accordance with another aspect of the invention, the method for characterizing analyte-specific affinity agents aforedescribed may further comprise repeating the provision of the surface, the exposure of the library of candidate affinity agents to the surface, and the causing of the analyte to contact the surface, and the analysis of the output fluid until the characterization of the analyte-specific affinity agents provides a desired threshold.

An analyte-specific affinity agent having affinity for an analyte that is identified or otherwise characterized according to the aforementioned methods constitutes an additional aspect of the invention.

In accordance with another aspect of the invention, an apparatus is provided for characterizing analyte-specific affinity agents having affinity for an analyte. The apparatus comprises an analyte receiver that receives the analyte. The apparatus further comprises a surface upon which a library of candidate affinity agents is adhered, the surface being in fluid communication with the analyte receiver so that the analyte contacts the surface when the analyte is received at the analyte receiver, which contact causes the analyte to contact the surface to thereby expose the library of candidate affinity agents adhered to the surface to the analyte, and which contact causes selected ones of the candidate affinity agents to react with the analyte to form reaction products, and to create an output fluid that comprises the reaction products. The apparatus further comprises an analyzer that analyzes the output fluid to characterize the analyte-specific affinity agents associated with the respective reaction products.

An analyte-specific affinity agent having affinity for an analyte that is identified or otherwise characterized by the aforementioned apparatus constitutes an additional aspect of the invention.

In accordance with another aspect of the invention, a method is provided for characterizing analyte-specific affinity agents having affinity for an analyte. The method comprises providing a library of candidate affinity agents wherein the library of candidate affinity agents comprises genomers. The method further comprises providing an analyte, and exposing the analyte to the library of candidate affinity agents to thereby characterize the analyte-specific affinity agents.

An analyte-specific affinity agent having affinity for an analyte that is identified or otherwise characterized according to the aforementioned method constitutes an additional aspect of the invention.

Preferably but optionally, the provision of the library of candidate affinity agents consists essentially of genomers. The provision of the library of candidate affinity agents, however, may include varying amounts of genomers, depending upon the application, for example, such as 1% genomers, 5% genomers, or 50% genomers. The genomers of the library of candidate affinity agents may comprise at least a portion of a human genome. The genomers may comprise a non-coding portion of the human genome, a coding portion of the human genome, or a combination of these.

The provision of the library of candidate affinity agents may comprise providing the library of candidate affinity agents as an array, for example, such as a microarray.

The provision of the analyte may comprise providing the analyte to comprise an analyte solution. The analyte also may be provided to comprise at least one of proteins, lipids, carbohydrates and toxins. The analyte also may be provided to comprise acetone, a volatile organic compound, a small molecule, at least one constituent of human breath, or a combination of these. In addition, the provision of the analyte may also comprise providing the analyte to comprise a target for a sensor application or a target for a therapeutic application.

In accordance with another aspect of the invention, an apparatus is provided for characterizing analyte-specific affinity agents having affinity for an analyte. The apparatus comprises an analyte receiver that receives the analyte. The apparatus further comprises a surface upon which a library of candidate affinity agents comprising genomers is adhered. The surface is in fluid communication with the analyte receiver so that the analyte contacts the surface when the analyte is received at the analyte receiver. This contact causes the analyte to contact the surface to thereby expose the library of candidate affinity agents adhered to the surface to the analyte, and the contact causes selected ones of the candidate affinity agents to react with the analyte to form reaction products, and to create an output fluid that comprises the reaction products. The apparatus further comprises an analyzer that analyzes the output fluid to characterize the analyte-specific affinity agents associated with the respective reaction products.

An analyte-specific affinity agent having affinity for an analyte that is identified or otherwise characterized by the aforementioned apparatus constitutes an additional aspect of the invention.

In accordance with yet another aspect of the invention, a method is provided for characterizing analyte-specific affinity agents having affinity for an analyte. The method comprises providing a library of candidate affinity agents wherein the library of candidate affinity agents comprises genomers and wherein the library of candidate affinity agents is attached to an array. The method further comprises providing an analyte, and exposing the analyte to the library of candidate affinity agents to thereby characterize the analyte-specific affinity agents.

Optionally, in the aforementioned method, the provision of the library of candidate affinity agents may comprise providing the library of candidate affinity agents attached to a microarray.

An analyte-specific affinity agent having affinity for an analyte that is identified or otherwise characterized according to the aforementioned method constitutes an additional aspect of the invention.

In accordance with yet another aspect of the invention, a method is provided for characterizing analyte-specific affinity agents having affinity for an analyte. The method comprises providing a library of candidate affinity agents wherein the library of candidate affinity agents comprises genomers, and further wherein the library of candidate affinity agents is attached to an array. The method further comprises providing an analyte, and exposing the analyte to the library of candidate affinity agents attached to the array. The method further comprises analyzing the array to thereby characterize the analyte-specific affinity agents.

An analyte-specific affinity agent having affinity for an analyte that is identified or otherwise characterized according to the aforementioned method constitutes an additional aspect of the invention.

The provision of the library of candidate affinity agents may comprise providing the library of candidate affinity agents attached to a microarray.

The analysis of the array to characterize analyte-specific affinity agents may further comprise measuring a signal, wherein analyte-specific affinity agents are characterized if the signal is different than a background. The signal may be at least one of an optical signal, an electrical signal, a thermal signal, and a mechanical signal. The optical signal may be fluorescence from the label. If it is an electrical signal, it may comprise at least one of a current, a voltage, a resistance, a capacitance, an inductance, and a combination thereof. The signal may be or comprise a thermal energy signal, for example, such as a radiation signal. It also may be or comprise a mechanical signal.

In accordance with yet another aspect of the invention, an apparatus is provided for characterizing analyte-specific affinity agents having affinity for an analyte. The apparatus comprises an analyte receiver that receives the analyte. It further comprises an array upon which a library of candidate affinity agents is attached, wherein the array is in fluid communication with the analyte receiver so that the analyte contacts the array when the analyte is received at the analyte receiver, which contact causes the analyte to contact the array to thereby expose the library of candidate affinity agents attached to the array to the analyte, and which contact causes selected ones of the candidate affinity agents to react with the analyte to form reaction products, and to create an output fluid that comprises the reaction products. The apparatus further comprises an analyzer that analyzes the output fluid to characterize the analyte-specific affinity agents associated with the respective reaction products.

The array may be or comprise a microarray.

An analyte-specific affinity agent having affinity for an analyte that is identified or otherwise characterized by the apparatus the aforementioned apparatus constitutes an additional aspect of the invention.

In accordance with yet another aspect of the invention, an apparatus is provided for characterizing analyte-specific affinity agents having affinity for an analyte. The apparatus comprises an array upon which a library of candidate affinity agents comprising a genomer is attached.

The array may be or comprise a microarray.

An analyte-specific affinity agent having affinity for an analyte that is identified or otherwise characterized by the aforementioned apparatus constitutes an additional aspect of the invention.

In accordance with yet another aspect of the invention, a method is provided for characterizing analyte-specific affinity agents having affinity for an analyte. The method comprises providing a library of candidate affinity agents wherein the library of candidate affinity agents comprises genomers. The method further comprises providing an analyte, and exposing the analyte to the library of candidate affinity agents to thereby cause selected ones of the candidate affinity agents to react with the analyte to form reaction products, where the reaction products are associated with certain analyte-specific affinity agents. The method further comprises segregating the reaction products from the remainder of the library of candidate affinity agents. The method further comprises amplifying the analyte-specific affinity agents associated with the reaction products to form an enhanced library of affinity agents to thereby characterize analyte-specific affinity agents.

An analyte-specific affinity agent having affinity for an analyte that is identified or otherwise characterized according to the aforementioned method constitutes an additional aspect of the invention.

A chimeric analyte-specific affinity agent having affinity for an analyte is yet another aspect of the invention. The chimeric analyte-specific affinity agent comprises at least two of the analyte-specific affinity agents characterized according to the method described herein.

Also or alternatively, a chimeric analyte-specific affinity agent may be or comprise at least two molecules, wherein at least one of the two molecules is an analyte-specific affinity agent characterized through methods described herein.

In accordance with yet another aspect of the invention, a kit is provided for characterizing analyte-specific affinity agents having affinity for an analyte. The kit comprises an analyte receiver that receives the analyte. The kit further comprises a surface upon which a library of candidate affinity agents is adhered. The surface is matable with the analyte receiver so that the analyte contacts the surface when the analyte is received at the analyte receiver. This contact causes the analyte to contact the surface to thereby expose the library of candidate affinity agents adhered to the surface to the analyte, and the contact causes selected ones of the candidate affinity agents to react with the analyte to form reaction products, and to create an output fluid that comprises the reaction products. The kit further comprises an analyzer for analyzing the output fluid to characterize the analyte-specific affinity agents associated with the respective reaction products.

In accordance with yet another aspect of the invention, a kit is provided for characterizing analyte-specific affinity agents having affinity for an analyte. The kit comprises a library of candidate affinity agents adhered to an array. The library of candidate affinity agents may comprise a genomer. Optionally, the kit may comprise a microarray.

### BRIEF DESCRIPTION OF THE DRAWINGS

The accompanying drawings, which are incorporated in and constitute a part of the specification, illustrate a presently preferred embodiments and methods of the invention and, together with the general description given above and the detailed description of the preferred embodiments and methods given below, serve to explain the principles of the invention.

FIG. 1 is a flow diagram that outlines and illustrates a presently preferred implementation of a method according to an aspect of the invention;

FIG. 2 is a flow diagram that outlines and illustrates a presently preferred implementation of a method according to another aspect of the invention;

FIG. 3 shows a presently preferred embodiment of an apparatus according to another aspect of the invention that may be utilized to identify analyte-specific affinity agents;

FIG. 4 shows another presently preferred embodiment of an apparatus according to an aspect of the invention that may be utilized to identify analyte-specific affinity agents;

FIG. 5 is a flow diagram that outlines and illustrates a presently preferred implementation of another method according to another aspect of the invention;

FIG. 6 is a flow diagram that outlines and illustrates a presently preferred implementation of a method according to still another aspect of the invention;

FIG. 7 is a flow diagram that outlines and illustrates a presently preferred implementation of a method according to still another aspect of the invention;

FIG. 8 shows another presently preferred embodiment of an apparatus according to an aspect of the invention that may be utilized to identify analyte-specific affinity agents;

FIG. 9 is a block diagram that outlines and illustrates a preferred implementation of a method according to still another aspect of the invention;

FIG. 10 shows an embodiment of a sensor according to another aspect of the invention that utilizes affinity agents to detect an analyte; and,

FIG. 11 shows another presently preferred embodiment of an apparatus according to an aspect of the invention that may be utilized to identify analyte-specific affinity agents.

### DETAILED DESCRIPTION OF THE PREFERRED EMBODIMENTS AND METHODS

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

In discussing the invention and its various embodiments and method implementations, the following terms are used, and should be construed in accordance with the following definitions and explanations.

A “peptide” as the term is used herein is used in its common usage to mean a combination of amino acids, amino acid residues or equivalent structures, joined by one or more peptide bonds. The peptide may or may not contain a secondary, tertiary or quaternary structure. The term “peptide” as used herein includes polypeptides and proteins.

A “nucleic acid” as the term is used herein is used according to its commonly understood meaning, and refers to a polymer comprising a sugar or sugar-like backbone coupled to any of the naturally occurring nucleic acid bases, including without limitation cytosine, guanine, uracil, adenine, thymine, inosine, and others together with combinations thereof, and modifications or substitutions of these naturally occurring nucleic acid bases. A nucleic acid may be single- or double-stranded. Examples of a nucleic acid include: DNA, RNA, and modified versions thereof, including those modifications that alter the chirality, the phosphodiester bond, the pentose, or the nitrogenous base. Other modifications that are known in the field may be used as well. Examples of nucleic acids further include: locked nucleic acids, peptide nucleic acids, those with phosphorothioate linkages, spiegelmers, and nuclease resistant nucleic acids. Nucleic acids also may be or comprise genomers.

A “genomer” refers to a nucleic acid or amino acid sequence that is found in nature, such as in living or once-living organisms. It can refer to either the entire sequence or a fragment thereof. Genomers include but are not limited to coding and non-coding, sense and anti-sense portions of DNA and RNA sequences and transcripted and/or translated versions thereof. They may be obtained via any number of methods including, but not limited to, isolating from nature, or producing synthetically or enzymatically. Genomers may be obtained from a number of locations within an organism, such as the nucleus, mitochondria, chloroplasts, viruses, free-flowing in body fluid, etc. Genomers may be shortened using digestion techniques, restriction enzymes, endonucleases, shearing (via, for example, temperature and/or pulling rapidly through a syringe), etc. Genomers may refer to the sequence as found in nature or in living or once-living organisms and/or they may include sequences that have altered secondary, tertiary, or quaternary structures.

An “affinity agent” as the term is used herein comprises or may be a nucleic acid or nucleic acid analog, a peptide, or a combination or combinations thereof; that reacts with an analyte, either entirely or in part, through non-Watson-Crick interactions.

A “library” as the term is used herein refers to the source from which to select an affinity agent. This source is comprised of different nucleic acids, peptides, and/or a combination or combinations thereof. The library may be isolated or synthesized chemically or enzymatically. The library may be or comprise genomers. A library can comprise or consist essentially of randomers, nonrandom sequences (such as naturally occurring sequences, patterns, or algorithmically generated sequences) or a combination thereof. For affinity agent selection methods that incorporate multiple selection rounds, the library size may change over time. Also, a library may include constituents of different sizes and/or lengths.

An “analyte” as the term is used herein is used according to its common meaning in the field to mean the molecule or species of interest. An analyte may be or comprise any of the following types of molecules, without limitation: small molecules (see definition herein below), organic compounds including volatile organics (see definition herein below), carbohydrates, proteins, amino acids, lipids, glycosylated biomolecules, toxins, therapeutic agents, whole cells, organisms, viruses, ions, complexes, other species, etc. In general, an analyte is the object of interest, regardless of composition. In certain cases, an analyte may comprise two or more different molecules or targets.

The term “small molecule” as used herein is one that is less than about 1,000 Daltons.

The term “volatile organic compound” as used herein is used according to its common meaning within the field of chemistry. A volatile organic compound may have a molecular weight less than 1000 Daltons.

An “array” refers to two or more affinity agents or candidate affinity agents linked to a substrate or spatially confined such that their composition is known by their location in either time and/or space, or by a distinguishing characteristic such as dye color, or by some combination of these strategies or by other strategies known in the field.

The term “adhere” as used herein is used according to its common use in the field and includes adherence to a surface through physical and chemical mechanisms whether in a specific or nonspecific fashion, such as through Van der Waals bonding, electrostatic bonding, adsorption, non-specific adsorption, hydrogen bonding, charge coupling, etc.

The term “adsorb” is used herein according to its common meaning in the field of chemistry, and includes physical adsorption, such as through Van der Waals bonding, electrostatic interactions, ionic interactions, polar interactions, hydrogen bonding, hydrophobic interactions, or a combination thereof, and chemical adsorption, involving chemical bonding to the surface, including bonding to a portion or constituent of the surface. Adsorption to a surface also can include adsorption of a species, such as the library, to other molecules, ions or the like attached to the surface. Nucleic acid affinity agents, for example, can exhibit affinity for random hexamers on a surface.

The term “attach” as used herein includes strong forms of attachment, such as covalent bonding, strong ionic bonding, adsorption, adhesion, entrapment, etc.

The term “label” as used herein is used generically in the sense of detection. The type of label used depends on the application. Examples are hereinafter provided. For laser-induced fluorescence, the label may be a fluorophore or other light emitting moeity. For electrochemistry, the label may be a current altering moeity. Some detection methodologies do not require a physical label (e.g., they are “label free”), such as surface plasmon resonance, carbon nanotubes, quartz crystal microbalance, etc. Thus, every reference to the term “label” should be interpreted as appropriate for the detection system.

The term “output fluid” as used herein is used according to its common meaning. For example, an output fluid is basically any fluid that comprises the reaction products. The output fluid may be withdrawn or otherwise outputted by a system, apparatus or method described herein. In one example, the output fluid may be the fluid in stagnant contact with a library of candidate affinity agents attached to an array. Or, the output fluid may be convected away from a library of candidate affinity agents. The output fluid may be any fluid phase, including a liquid or gas phase and as described later herein.

Currently, genomers have not received much attention as ligands. This lack of attention is due to several commonly held beliefs. First, few non-Watson-Crick interactions are expected from genomers. This is due in part to the environment in which most genomers are found. The large degree of secondary, tertiary and quaternary structure in genomes or gene products may prevent such interactions from forming. Further, compartmentalization of genomes and gene products (e.g., nucleus versus cytosol) may prevent them from having access to a wide variety of potential interactions.

When genomes are digested or portions of genes are chemically synthesized, however, the higher order structures are for the most part removed. Moreover, use of genomers outside of their native environment provides an opportunity to discover interactions that would not have otherwise been known. Thus, by removing secondary structures and/or by forcing contact with nonnative substances, a variety of potential ligands may be discovered.

This discovery can be further facilitated by the use of arrays. In large measure due to the human genome project, array technologies have accelerated and some companies are contemplating putting the entire human genome on an array (more than three billion features). This large number of features approaches the size of combinatorial libraries. Thus, the large number of features in combination with the ever more ubiquitous gene arrays brings forth the possibility of a set of affinity agents not previously described by aptamers or SELEX methodologies. Thus, there is a need for a new definition for affinity agents and methods for identifying them.

In accordance with one aspect of the invention, a method is provided for characterizing analyte-specific affinity agents having affinity for an analyte or analytes. The analyte, as noted herein above, is the object of interest, regardless of composition. It may be or comprise, for example, a target for a sensor application. An example would include one or more constituents of human breath, for example, such as acetone. The analyte also may comprises or be a target for a therapeutic application. The analyte may comprise, for example, at least one of proteins, lipids, carbohydrates, and toxins.

The analyte preferably is in the fluid phase, including a gas phase (including vapor phase), a liquid phase, mixed phases, aerosols, suspensions, etc. The analyte preferably, and typically, is provided as an analyte solution. An “analyte-specific” affinity agent is an affinity agent (including a candidate affinity agent) that has affinity for the specific analyte or analytes of interest in a particular application or use. If a particular apparatus or method according to the invention is being used to detect acetone in a sensor application, for example, analyte-specific affinity agents would include those affinity agents that have affinity for acetone.

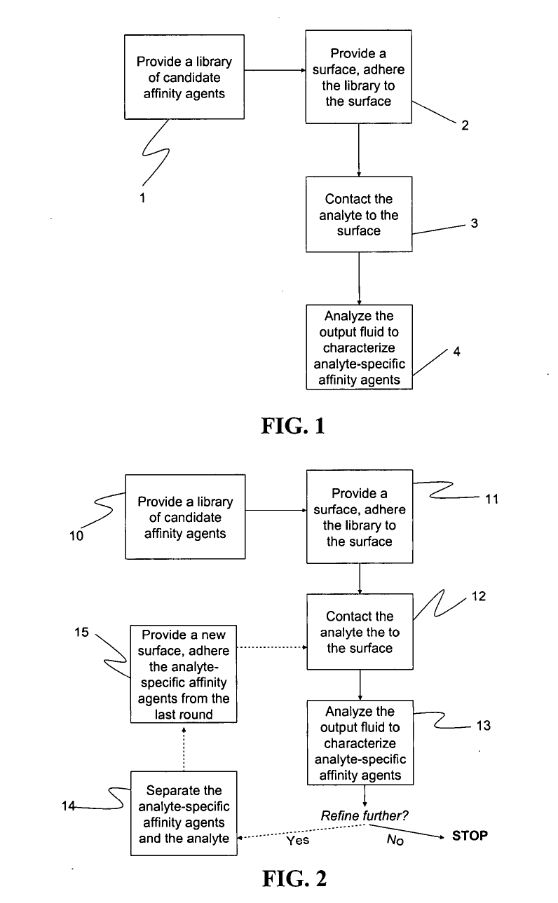

A presently preferred but merely illustrative implementation of this method is outlined and illustrated in FIG. 1.

The method according to this aspect of the invention comprises providing a library of candidate affinity agents. As noted herein above, the affinity agents comprise or may be a nucleic acid or a peptide, or a combination or combinations thereof, that react with an analyte, either entirely or in part, through non-Watson-Crick interactions. The method according to this aspect of the invention aids in identifying affinity agents that have affinity specifically for the analyte or analytes of interest. In presently preferred implementations, one typically begins with a library that includes a relatively large number of agents that might or might not have satisfactory affinity for the analyte or analytes. Accordingly, this population of agents is referred to herein as “candidate” affinity agents. They represent candidates in the sense that one suspects they may have the desired affinity, but they must be evaluated according to methods such as those described herein to confirm or disprove this.

The library of candidate affinity agents may comprise at least one nucleic acid. The nucleic acid or nucleic acids may comprise non-naturally occurring nucleic acids and/or naturally occurring ones. In a presently preferred embodiment, for example, the library comprises randomers of between 5 and 1000, 5 and 500, 10 and 200, 20 and 70 base pairs in length.

For example, a library of affinity agents that is synthesized may be given by: 5′ATACCAGCTTATTCAATT-N60-AGATAGTAAGTGCAATCT-3′. This library may be purified via PAGE and labeled via a fluorescent tag. This library may, for instance, be used to identify an analyte-specific affinity agent having affinity for acetone.

The library of candidate affinity agents also may comprise a peptide, i.e., one or more peptides. The library of candidate affinity agents further may comprise a genomer, which preferably but optionally may comprise the human genome, or at least a portion of it. The human genome used in the library may comprise one or more coding portions of the human genome, one or more non-coding portions of the human genome, or combinations of these. The genomer also may comprise mouse genome and/or the genome of other organisms. It also may comprise or consist of or essentially of messenger RNA.

The library may be labeled or coupled to a second molecule. In certain embodiments, it may be preferred for the library to be labeled with a fluorescent tag. In certain embodiments, the library may also be biotinylated. A handle, such as a primer sequence, also may be added for manipulation of the sequences in the library.

The method according to this aspect of the invention further comprises providing a surface suitable for adhesion of the library of candidate affinity agents, and exposing the library of candidate affinity agents to the surface to thereby adhere the library of candidate affinity agents to the surface. The surface according to this aspect of the invention serves the primary function of supporting or immobilizing the candidate affinity agents so that they may be contacted by the analyte or analytes. Suitable surfaces may include, for example, a flat plate, spheres or beads, a rough surface, porous or tortuous substrates, fibrous networks, channeled surfaces, and the like.

The surface preferably but optionally comprises a nonspecific surface, i.e., it generally adsorbs most if not all molecules of a particular class or molecules that share a certain characteristic or set of characteristics, which are exposed to it. A nonspecific surface may be or comprise a surface that is coated with a chemical or otherwise treated in a manner that renders the surface “nonspecific.” A nonspecific surface also may be a combination of two or more materials or surface types. A nonspecific surface may be identified for at least a portion of the molecules that comprise the library. This nonspecific surface then can be expected to adsorb substantially the entire library or at least a sufficient portion of it. An example of a nonspecific surface would be a silica, for example, such as silica surface or silica beads. The surface also may be or comprise a spin column. In a presently preferred implementation of the present method, for example, the surface comprises silica beads to which candidate affinity agents in the form of nucleic acids adsorb nonspecifically.

It was noted herein above that the surface may comprise another molecule, ion or the like attached to the surface. As an example, in another preferred implementation, the surface comprises random hexamers or other randomers immobilized on a substrate or directly onto the surface, to which affinity agents, such as nucleic acids, are adsorbed.

The exposure of the library of candidate affinity agents to the surface to adhere the library to the surface may be carried out in any of a number of ways. In presently preferred implementations of the method according to this aspect of the invention, the exposure of the library of candidate affinity agents to the surface comprises adsorbing the library to the surface nonspecifically, so that most and preferably all of the candidate affinity agents are adsorbed to the surface.

The manner of exposing the library to the surface is not necessarily limiting, and may include, for example, convection, incubation, stagnant contact, or by other means known in the field. This exposure may require certain conditions depending on the application and the desired results, such as regulated temperature, pH, ion concentration, etc. Additionally, this exposure may require the presence of certain reagents or solutions, such as a solution with high salt concentration.

If a spin column is used, the library may be loaded onto the spin column and adhered to silica via adsorption techniques using a Qiagen QIAquick Nucleotide Removal Kit (the columns in this kit typically bind nucleic acids between 17 and 10 kb in length).

When the library is exposed to the surface, the library adheres to the surface, which includes such interactions as nonspecific adsorption, hydrogen bonding, charge coupling, etc.

An analyte may be provided in a number of manners. For instance, the analyte may be provided via forced expiration of human breath containing the analyte, a pump or micropump, releasing drops containing the analyte from a pipette, capillary action, etc.

As noted herein above, the analyte is the object of interest. There may be one analyte or more than one. It may be or comprise any number of different molecules, ions, complexes or other species. The analyte also may comprise small molecules, such as ethanolamine, and/or volatile organic compounds, such as acetone, ethanol, etc. Presently preferred implementations of the method according to this aspect of the invention as more fully described herein below are particularly well suited for identifying affinity agents for these types of analytes, for example, because they do not require alteration of the analyte itself (e.g., changing the structure, labeling, etc). However, this method may be employed for analytes in general.

The analyte or analytes may comprise at least one of, or some combination of, a peptide, a lipid, a carbohydrate, a toxin, an enzyme, and a catalyst.

The analyte comprises or is otherwise within a fluid (e.g., a liquid, gas, vapor, aerosol, suspension, etc.), and preferably comprises a solution comprising the analyte or analytes and a solvent, working fluid or carrier.

As noted herein above, the method according to this aspect of the invention is useful for such applications as sensor applications and therapeutic applications. Accordingly, the analyte or analytes may comprise targets for such applications. The method is useful, for example, for human breath analysis, wherein the analyte or analytes preferably would comprise one or more constituents of human breath. Examples of breath constituents that may serve as analytes may be or comprise, without limitation: acetone, ethanol, acetaldehyde, isoprene, pentane, ethane, alkanes, benzene, carbon-13, methanol, leukotrienes, hydrogen peroxide, isoprostane, peroxynitrite, cytokines, glycans, carbon monoxide, chloroform, dichlorobenzene, trimethyl amine, dimethyl amine, diethyl amine, methanethiol, methylethylketone, o-toluidine, pentane sulfides, hydrogen sulfide, sulfated hydrocarbons, cannabis, G-HBA, nitric oxide, propane, butane, other ketones, ethyl mercaptane, dimethyl sulfide, dimethyl disulfide, carbon disulfide, 3-heptanone, 7-methyl tridecane, nonane, 5-methyl tridecane, 3-methyl undecane, 6-methyl pentadecane, 3-methyl propanone, 3-methyl nonadecane, 4-methyl dodecane, 2-methyl octane, trichloroethane, 2-butanone, ethyl benzene, xylene (M, P, O), styrene, tetrachloroethane, toluene, ethylene and hydrogen.

Examples of other analytes may be or comprise, without limitation: HIV protease, GP120, GP41, glucose, HIV nucleocapsid, albumin, alpha-1 acid glycoprotein, antibodies, IgG, insulin, CD4 receptor, type A influenza, type B influenza, glycans, glycoconjugates, 3′ sialyl lactose, 6′ sialyl lactose, bromobenzene, bromochloromethane, bromodichloromethane, bromoform, bromomethane, 2-butanone, n-butylbenzene, sec-butylbenzene, tert-butylbenzene, carbon disulfide, carbon tetrachloride, chlorobenzene, chloroethane, chloroform, chloromethane, 2-chlorotoluene, 4-chlorotoluene, dibromochloromethane, 1,2-dibromo-3-chloropropane, 1,2-dibromoethane, dibromomethane, 1,2-dichlorobenzene, 1,3-dichlorobenzene, 1,4-dichlorobenzene, dichlorodifluoromethane, 1,1-dichloroethane, 1,2-dichloroethane, 1,1-dichloroethene, cis-1,2-dichloroethene, trans-1,2-dichloroethene, 1,2-dichloropropane, 1,3-dichloropropane, 2,2-dichloropropane, 1,1-dichloropropene, cis-1,3-dichloropropene, trans-1,3-dichloropropene, ethylbenzene, hexachlorobutadiene, 2-hexanone, isopropylbenzene, p-isopropyltoluene, methylene chloride, 4-methyl-2-pentanone, methyl-tert-butyl ether, naphthalene, n-propylbenzene, styrene, 1,1,1,2-tetrachloroethane, 1,1,2,2-tetrachloroethane, tetrachloroethene, toluene, 1,2,3-trichlorobenzene, 1,2,4-trichlorobenzene, 1,1,1-trichloroethane, 1,1,2-trichloroethane, trichloroethene, trichlorofluoromethane, 1,2,3-trichloropropane, 1,2,4-trimethylbenzene, 1,3,5-trimethylbenzene, vinyl acetate, vinyl chloride, xylenes, dibromofluoromethane, toluene-d8, and 4-bromofluorobenzene.

In some applications it may be desirable to adjust the stringency of the analyte fluid, and different stringency levels can be used in different trials or applications. The stringency level normally would be higher if the concentration of analyte in solution is lower, or when denaturing solvents are present that decrease the affinity of the library for the analyte. The stringency also normally would be higher when ions or solvents are present that stabilize the affinity of the library for the substrate, or for higher temperatures.

Preferably but optionally, the material in which the analyte is dissolved is not an interfering substance. For example, if the analyte is acetone and the application is breath analysis, then it may be desirable for the acetone to be dissolved in a non-ethanol environment, because ethanol is an interfering substance in breath analysis of acetone. In this example, acetone may be dissolved in an alternative solvent, for example, such as water. Acetone itself also may be “solution.” The analyte may comprise or constitute glucose, preferably dissolved in water.

The method according to this aspect of the invention also comprises causing the analyte to contact the surface to thereby expose the library of candidate affinity agents adhered to the surface to the analyte, to cause selected ones of the candidate affinity agents to react with the analyte to form reaction products, and to create an output fluid that comprises the reaction products.

One may cause the analyte to contact the surface in a variety of ways. One way is to flow or convect the analyte or analyte solution across the surface to which the library is adhered. This contacting may be facilitated by gravity, by centrifugal force, or by use of a pump or the like. If the library is adsorbed to silica beads of a DNA spin column, for example, the analyte in solution may be introduced to the top of the spin column and the fluid output collected at the bottom of the column. If the library is adhered to a flat plate, the plate may be placed at an angle with respect to, for example, a level work bench, the analyte or analyte solution may be passed at the top of the flat plate, and the output fluid may be collected at the bottom of the plate. The library also may be adsorbed or otherwise adhered to the channels of a microfluidic device. In this instance, for example, the analyte or analyte solution is propelled via a micropump through the microfluidic channels, and the output fluid is collected in a compartment following the library-coated channels.

Instead of the analyte being convected across the library adhered to a surface, the library adhered to a surface may be moved through a solution containing the analyte. For example, the surface to which the library is adhered may be attached to a micromanipulator, the micromanipulator may expose the library to the analyte in solution, and the library may then be removed after a designated period of time. In another approach, the library may be adhered to a rod or probe that is coated with a material to which the library adsorbs, and this probe may be moved around in the fluid phase that contains the analyte or analytes.

The library also may be adhered to the surface of magnetic beads, and these beads can be mixed with solution and the beads extracted after sufficient hybridization time. Similarly, the library can be adhered to silica beads, which are allowed to mix with the analyte in free solution. Following mixing, the beads are filtered out and the output fluid collected.

As the analyte-containing fluid flows over or otherwise contacts the surface and the library adheres to it, the analyte will react in a general sense with the candidate affinity agents. “Reaction” as the term is used here is used in the general sense to include not only chemical reaction (e.g., ionic or covalent bonding), but also catalysis, chemical modification, alterations to primary, secondary, tertiary, or quaternary structures, Van der Waals interactions, and adsorption or other physical reactions or interactions such as binding.

The result of the reaction is the creation of “reaction products.” These reaction products may include conformation changes to constituents of the library, actual reaction products from a chemical reaction, complexes from physical interactions such as a binding complexes, and the like. For example, if the library comprises nucleic acids and the analyte is a protein, an example of a reaction product may be a nucleic acid that has undergone a conformational change and which is bound to the protein. Other examples are, of course, possible.

These reaction products generally disperse into the analyte-containing fluid and become a part of the “output fluid.” This output fluid may be analyzed directly, or it may be collected for analysis. Similarly, it may be analyzed intact, or it may be separated into components or otherwise modified for analysis.

The method according to this aspect of the invention further comprises analyzing the output fluid to characterize the analyte-specific affinity agents associated with the respective reaction products.

The term “characterize” as used herein is used according to its broad but common meaning within the field and includes obtaining information about the analyte-specific affinity agent or agents. For example, characterizing the analyte-specific affinity agents may involve identifying the presence of the affinity agent, completely or partially determining its chemical makeup (e.g., sequencing a nucleic acid), isolating, determining certain characteristics of the analyte-specific affinity agent, ascertaining or estimating its concentration, reactivity, and the like. Characteristics that may be important include, but are not limited to, size, charge, the presence of certain functional groups, etc. Size, for instance and in certain implementations, may be determined by gel electrophoresis. Other manners of identifying an affinity agent may be used as well.

The affinity agent can be characterized by determining the chemical makeup of the analyte-specific affinity agent. This may occur, for example, by sequencing the sample directly or by amplifying the affinity agents in the output fluid and sequencing them, depending on the concentration of the affinity agent in the original library and the sequencing needs.

In attempting to characterize the affinity agent, the analysis of the output fluid may comprise separating the analyte-specific affinity agents from the analyte. This may or may not be necessary or desirable. For example, if characterizing the affinity agent involves determining the size of the affinity agent, separation from the analyte may not be necessary. If characterizing the affinity agent involves determining its chemical makeup and if determining its chemical makeup requires amplification, then it may or may not be necessary to separate the affinity agent from the analyte, depending on the method of amplification used and the nature of the analyte. As may be appreciated, there are various methods of amplification (including those described herein and others known in the field) that may be used, which under certain circumstances, may aid in characterizing the affinity agent.

The analysis may take a variety of forms, depending upon such factors as the specific analyte or analytes, the library used, the flow and reaction regimes, etc. The analysis may comprise, for example, isolating the analyte specific affinity agents, sequencing them, and the like.

In optional but presently preferred implementations of this aspect of the invention, the analysis of the output fluid comprises using differential binding. The analysis of the output fluid also may comprise a comparison of a library-silica affinity to a library-analyte affinity.

Differential binding is a method of analysis that allows for recognizing that affinity agents have certain characteristics. One such characteristic is comparative binding affinities where the affinity of the analyte-specific affinity agent is greater than the affinity of the affinity agent to a reference such as a surface. For example, if the surface is silica beads, then the affinity agent may be selected to have a greater affinity to the analyte than to the silica beads. Thus, in this example, differential binding has utility in that the threshold of acceptable affinity for the analyte-affinity agent interactions is compared against a reference affinity. If the reference is particularly important (e.g., the reference coats or is the material of the conduits within which the analyte-specific affinity agent is likely to pass), then this example of a use of differential binding is highly advantageous.

The analysis of the output fluid may involve determining approximately how many analyte-specific affinity agents remain in the output fluid. This may be helpful, for example, in determining if further refinement of the analyte-specific affinity agents in the output fluid is necessary. In these instances, it may be desirable to label the library with a fluorescent tag and measure the fluorescent intensity in the output fluid. The concentration of affinity agents in the output fluid also may be determined by other methods, such as, for example, absorbance. In any case, the concentration of affinity agents resulting in the output fluid may indicate or dictate whether further refinement and accordingly another iteration of selection is needed.

Specificity may be important in some applications, in addition to affinity. “Specificity” is used herein in its broad but common meaning in the field of bioengineering. For example, a specific interaction occurs when an analyte-specific affinity agent interacts with the analyte in a manner that is distinguishable, exclusive in whole or in part, or preferred. Specificity may be determined, for example, by comparing characteristics of the analyte-specific affinity agent interaction with reference parameters or with characteristics of an interfering substance-affinity agent interaction.

Certain implementations yield high-affinity and high-specificity affinity agents. For example, the user may pass a solution containing an interfering substance across the library adhered to a surface during the selection procedure. In these cases, the affinity agents in the output fluid preferably are discarded because they have an affinity for the interfering substance. The same surface also is exposed to the analyte in solution as described earlier in this document. The output fluid collected from this exposure round results in affinity agents with relatively high affinity for the analyte and relatively low affinity for the interfering substance. Multiple iterations of this method with selection and counter-selection in any order or arrangement and of the appropriate stringency are, of course, possible.

There is a possibility that the output fluid will not contain an affinity agent with affinity to the analyte. This may occur in the event that the analyte is the limiting reagent in the analyte-affinity agent binding process. The investigator would need to determine this, depending on the application. However, in most cases, the energy change from the interaction of the affinity agent with the analyte will result in a conformational change to the affinity agent. This conformational change will, in most cases, overcome the energy of interaction between the affinity agent and the surface.

Generally, the output fluid contains affinity agents with affinity to the analyte or analytes. If the number of different affinity agents in the output fluid is sufficient, only one round of the preferred method implementation needs to be performed. The output fluid containing the affinity agents in this sample are analyzed as generally described herein above. If the number of different affinity agents in the output fluid is unsatisfactorily high (e.g., >1000) or otherwise preferably would be lower, however, a modification or variation of the method implementation as generally described herein above may be employed, wherein multiple (two or more) rounds of analyte-candidate affinity agent contacting are required.

Accordingly, a method for such multi-round processing according to another aspect of the invention will now be described. The method comprises performing the method as described herein above, comprising providing a library of candidate affinity agents, providing a surface suitable for adhesion of the library of candidate affinity agents, exposing the library of candidate affinity agents to the surface to thereby adhere the library of candidate affinity agents to the surface, causing the analyte to contact the surface to thereby expose the library of candidate affinity agents adhered to the surface to the analyte, to cause selected ones of the candidate affinity agents to react with the analyte to form reaction products, and to create an output fluid that comprises the reaction products, and analyzing the output fluid to characterize the analyte-specific affinity agents associated with the respective reaction products. Each of these is described herein above.

The method further comprises separating the analyte-specific affinity agents from the analyte, providing a second surface suitable for adhesion of the analyte-specific affinity agents, exposing the analyte-specific affinity agents to the second surface to thereby adhere the analyte-specific affinity agents to the second surface, causing the analyte to contact the second surface to thereby expose the analyte-specific affinity agents adhered to the second surface to the analyte, to cause selected ones of the candidate affinity agents to react with the analyte to form second reaction products, and to create a second output fluid that comprises the second reaction products, and analyzing the second output fluid to identify enhanced analyte-specific affinity agents associated with the respective reaction products. A presently preferred implementation of this method is outlined in FIG. 2.

When multiple rounds are to be performed, the affinity agents from the output fluid preferably are released from the analyte via a suitable process appropriate for the analyte-affinity agent combination, and then reloaded onto the surface or another surface for repeat exposure to the analyte or analyte solution. This can be accomplished in a number of ways.

If the analyte is a volatile organic compound, it is possible that the analyte will evaporate if allowed sufficient time and thereby dissociate from the complex. Alternatively, the analyte-affinity agent interaction may be disrupted via changes in the temperature, buffer solution, pH, etc. Or, if the library is biotinylated, then the analyte-affinity agent bond may be disrupted if the reaction product (e.g., the analyte-affinity agent binding complex) is passed across a streptavidin-coated surface to which the affinity agents preferentially bind. If the biotinylation approach is utilized, the affinity agents preferably are released from the streptavidin surface via a process such as heat elution, wash steps, or other means of disrupting the biotin-streptavidin bond as known in the field.

In yet another example, the analyte itself is biotinylated and the analyte-affinity agent bond may be disrupted if the reaction product (e.g., the analyte-affinity agent binding complex) is passed across a streptavidin-coated surface to which the biotinylated analyte preferentially binds. This yields an analyte, which may be part of a reaction product, that is bound to a surface. In this example, the affinity agents may be released from the surface-bound analyte via a process such as heat elution, wash steps, or other means of removing an affinity agent from a surface bound molecule

In providing the second surface suitable for adhesion of the analyte-specific affinity agents, the selection criteria and other comments set forth herein above regarding the surface for adhesion of the library apply to it as well. Thus, the second surface may be identical to the surface described in connection with the first round of the preferred method implementation. Indeed, the second surface may be or comprise the first surface, in original or washed or otherwise modified form, as described herein above, so that only a single surface is used as both the first and second surface. The second surface may also be different from the first, providing additional screening properties, such as a higher affinity for the library in order to create a higher stringency.

The exposure of the analyte-specific affinity agents to the second surface to adhere the analyte-specific affinity agents to the second surface may be carried out as described herein above with respect to exposure of the library of affinity agents to the first surface.

The exposure of the analyte or analytes to the second surface preferably comprise providing the same analyte or analytes that are used in the first round. As described herein above, it preferably comprises providing an analyte solution that includes the one or more analytes of interest.

When multi-round processing is performed, it may be desirable to increase the stringency of the exposure or decrease the concentration of analyte in solution. The appropriate stringency level may vary depending on the analyte and the nature of the application. For an analyte comprising a protein, for example, in some implementations of the method, stringency levels may vary (low to high) between 1M and 1 fM, 1M and 1 nM, 1M and 1 uM, 1 mM and 1 pM, and 1 uM and 1 nM.

The manner in which the analyte is caused to contact the second surface also may be as described herein above with respect to the first pass of the preferred method implementation.

The contacting of the analyte-specific affinity agents adhered to the second surface with the analyte solution is carried out so that it causes analyte-specific affinity agents (at least a portion of them, e.g., such as a particular type of class of analyte-specific affinity agents) to react with the analyte to form second reaction products, and to create a second output fluid that comprises the second reaction products.

The approaches for contacting the library of affinity agents as described herein above also apply to contacting the analyte-specific affinity agents with the analyte in this later-round context. As contact occurs and binding or other reaction occurs, the second reaction products are produced. The classification of the second reaction products preferably is the same as the reaction products described herein above (e.g., binding complexes, alterations to secondary structure, etc). These second reaction products diffuse into or otherwise move into the bulk fluid, which becomes the second output fluid as the reactions proceed and the second reaction product concentration increases (the concentration in this specific reference is to the concentration of overall second reaction product, understanding that the constituents of the second reaction product may differ from one another). The second output fluid thus preferably has the same general composition as the output fluid described herein above, but wherein the second reaction products are associated with affinity agents of greater affinity for the analyte.

The analysis of the second output fluid to identify enhanced analyte-specific affinity agents associated with the respective reaction products may be carried out as described herein above for the analysis of the output fluid.

The number of rounds to be performed may vary, depending on the application, the analyte, and other factors. The exposure of the library of candidate affinity agents to the surface, the causing of the analyte to contact the surface, and the analysis of the output fluid may be repeated until the characterization of the analyte-specific affinity agents provides a desired threshold. In certain implementations, for example, the number of rounds may be between 1 and 5, 2 and 5, 1 and 10, 1 and 20, and 5 and 20. In some implementations, amplification of the library between rounds may be desirable. For example, polymerase chain reaction (PCR) can be used to amplify libraries composed of nucleic acids.

In some instances, if the concentration of affinity agents in the output fluid is low, it may be necessary or appropriate to amplify remaining sequences before beginning another round. But, this is an optional step. It may or may not be necessary, depending on the application. Alternatively, it is possible to begin with an original library which contains duplicate copies of certain or all of the constituents in order to verify that each member of the library has adequate representation to provide confidence in the results. One implementation of this concept might include a 20mer randomer used in a micromole-scale synthesis to produce an estimated 1,000 copies of each possible sequence. Alternatively, the test can be performed multiple times to evaluate for statistical significance in selection of a given sequence.

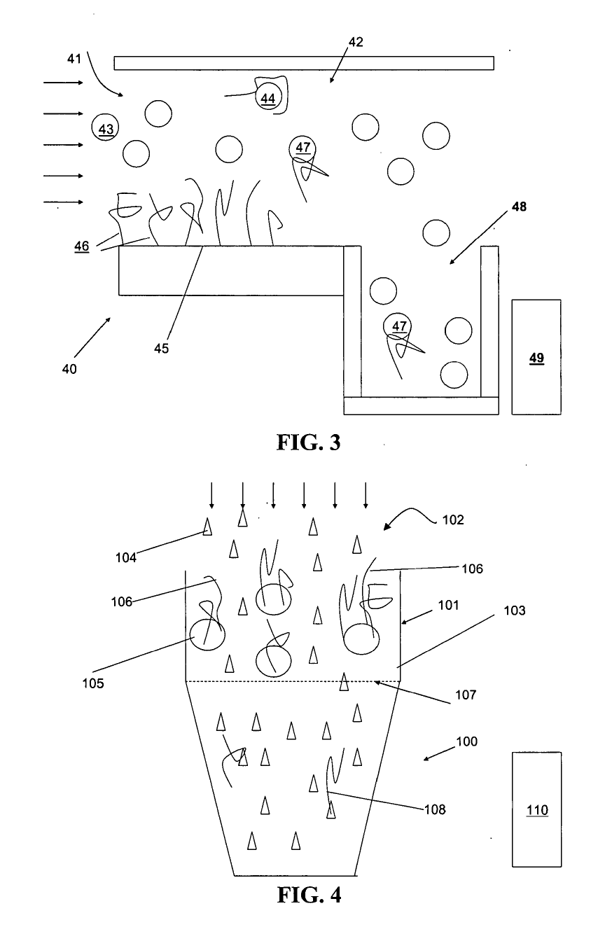

In accordance with another aspect of the invention, an apparatus is provided for characterizing analyte-specific affinity agents having affinity for an analyte. For illustrative purposes and to better explain the principles of the invention, this apparatus will be described with reference to and in connection with a presently preferred but merely illustrative embodiment of this aspect of the invention in the form of a benchtop analyzer 40, as shown in FIG. 3.

The apparatus comprises an analyte receiver that receives the analyte. Although this analyte receiver may take any of a number of forms, depending upon the specific application, in benchtop analyzer 40, this analyte receiver comprises an input orifice 41 that is coupled to or is otherwise in fluid communication with an analyte source (not shown). In the event that the analyte is a constituent of human breath, the input orifice may be tubing connected to a simulated breath system (not shown), a mouthpiece (not shown) or breathe gas reservoir (not shown). Input orifice 41 is coupled to or otherwise is in fluid communication with a chamber 42 so that, when the analyte or analytes 43, alone or in a solution, but in fluid form, are inputted into the orifice 41, the fluid passes into chamber 42.

The apparatus according to this aspect of the invention also comprises a surface upon which a library of candidate affinity agents is adhered. The surface and the library of candidate affinity agents may be any of those identified herein above. In benchtop analyzer 40, a surface 45 which comprises silica is disposed within chamber 42. Surface 45 more specifically comprises a lower wall of chamber 42 and is in fluid communication with input orifice 41 so that the analyte 43 contacts the surface when the analyte is received at and passes through orifice 41. The library of candidate affinity agents 46 adhered to surface 45 comprises genomers that are between 20 and 200 bases in length.

When the analyte contacts the surface, for example, which occurs when the fluid containing the analyte is passed into and through the orifice 41, the library of candidate affinity agents 46 adhered to surface 45 are exposed to analyte 43. This contact causes selected ones of the candidate affinity agents, those that have true affinity for the analyte, to react with the analyte to form reaction products 47. These reaction products move into the bulk fluid. Assuming that the amount of fluid and analyte is maintained as a constant, as the analyte in this fluid is consumed and, therefore, as the analyte concentration decreases (again assuming that the analyte input is not replenished), and as the concentration of the reaction products in the fluid increases, the fluid becomes the output fluid, as has been described herein above. As stated earlier, however, the fluid merely needs to contain reaction products to be characterized as the output fluid. In certain cases, the reaction products may be the analyte-specific affinity agent itself if, for example, the analyte-affinity agent interaction caused a conformational change in the affinity agent that caused its release from the surface.

Benchtop analyzer 40 further comprises an output orifice 48 into which the output fluid flows. The output orifice may comprise any structure that is capable of containing or otherwise providing the output fluid for collection or analysis. For example, an output orifice may be a needle-port, a beaker, the bottom portion of a spin column, or a connection to the output analyzer 49. In analyzer 40, output orifice 48 comprises a chamber in which the output fluid is collected.

The apparatus also preferably but optionally comprises an output analyzer 49. As stated herein above, analysis of the output fluid may comprise any form of analysis that is appropriate for the application. Example of analysis of the output fluid as described herein include: isolating or identifying the analyte-specific affinity agents, recognizing characteristics of the analyte-specific affinity agents, approximating or determining the number of distinct analyte-specific affinity agents, etc. With respect to the benchtop analyzer, the output analyzer may be any contrivance configured to analyze the output fluid as specified herein. For example, it may be an ultraviolet (UV) spectrophotometer, a chromatograph, a DNA sequencer, a mass spectrometer, a mathematical model or software algorithm that operates in conjunction with data obtained from the experiment, a microscope, user observation coupled with knowledge of how certain molecular entities behave, or any combination thereof. The output analyzer may be partly or fully physically attached to the benchtop analyzer and/or it may require a sample of the output fluid.

In this embodiment, output analyzer 49 analyzes the output fluid using a combination of differential binding and sequencing to identify the analyte-specific affinity agents associated with the respective reaction products.

Another embodiment according to this aspect of the invention in the form of a portable analyzer 100 is shown in FIG. 4. Portable analyzer 100 comprises a DNA spin column 101 that comprises an input orifice 102 and a chamber 103 that receive one or more analytes 104. A surface is disposed with spin column 101 in the form of a plurality of silica beads 105. A library of candidate affinity agents 106 is adsorbed to silica beads 105. Portable analyzer 100 further comprises an output orifice 107 in fluid communication with the chamber 103.

When a fluid comprising the analyte 104 is passed through DNA spin column 101, the analyte reacts with selected ones of the candidate affinity agents to create reaction products 108. These reaction products 108 move into the fluid, which becomes an output fluid as described herein, and that fluid moves through the output orifice 107 and into a collection vesicle. The output fluid may be analyzed using an output analyzer 110. The analysis of the output fluid may be by any of the methods described herein such as, for example, sequencing to identify the analyte-specific affinity agents and use of a spectrophotometer.

The differential binding described herein usually can be modeled mathematically. For example, the binding affinity of the analyte to the affinity agent in most cases is greater than the affinity of the affinity-agent or library to the surface. In some embodiments, e.g., as described herein above, the surface may be or comprise silica. In such embodiments, the library-silica binding affinity may be characterized and pre-determined such that the minimum binding affinity of the analyte to the affinity agent may be approximated. If the affinity agents are identified using a DNA spin column, then these principles may be applied and may also include reaction kinetic considerations similar to those used for a packed bed reactor.

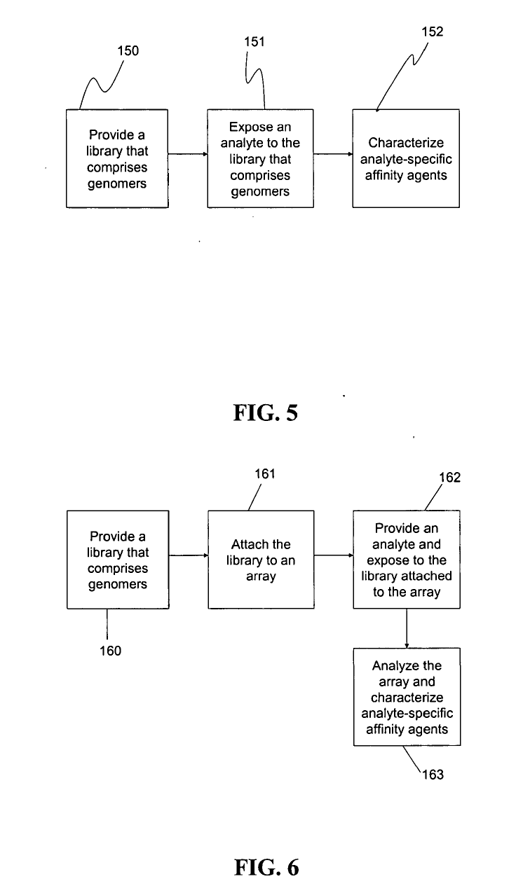

In accordance with another aspect of the invention, a method is provided for characterizing analyte-specific affinity agents having affinity for an analyte. A presently preferred implementation of this method is outlined in FIG. 5

The method according to this aspect of the invention comprises providing a library of candidate affinity agents wherein the library of candidate affinity agents comprises genomers.

In certain implementations, the library of candidate affinity agents may consist of or consist essentially of 1%, 5%, 50% genomers. In a preferred implementation, the library of candidate affinity agents consists essentially of genomers. These genomers preferably but optionally comprise at least a portion of a human genome, and preferably but optionally they comprise a coding portion of the human genome. In another embodiment, they may also comprise non-coding elements of the human genome.

The method also comprises providing an analyte, and exposing the analyte to the library of candidate affinity agents to thereby identify the analyte-specific affinity agents. As stated earlier herein, analyte is used in its broad, but common meaning within the field. Generally, an analyte is the molecule/s or species of interest. Analytes such as those described herein above may be used.

In accordance with still another aspect of the invention, a method is provided for characterizing analyte-specific affinity agents having affinity for an analyte. A presently preferred implementation of this method is outlined in FIG. 6.

The method comprises providing a library of candidate affinity agents wherein the library of candidate affinity agents comprises genomers, and further wherein the library of candidate affinity agents is attached to an array. The library of candidate affinity agents may comprise those genomers described herein above. Different genomes can be used and virtually any genome is an acceptable source for a genomer. Examples of such genomes, without limitation, include: human, mouse, rice, honey bee, Arabidopsis thaliana, virus phage X174, fruit fly, puffer fish, E. coli, bacteriophage MS2, Amoeba dubia, Populus trichocarpa, minimal genomes, etc. Human genome arrays may be used for this method (for example and without limitation, a 1×244 k from Agilent Technologies). Also, different genomes can be combined on an array. For instance, a single array may contain genomers derived from the human genome and also the mouse genome.

The library of candidate affinity agents that comprises genomers may be attached to the array in a number of different ways. The attachment may be via covalent bonds, such as through amine functionalization, free radical polymerization, thioether linkages, and others known to those skilled in the art. Attachment may also be in the form of non-covalent, but strong interactions such as that occurring between biotin and streptavidin. In some embodiments, the array is synthesized in situ, such as with light directed or electrode activated synthesis. Many other methods for attaching nucleic acids to arrays are also known in the field.

The method according to this aspect of the invention also comprises providing an analyte, and exposing the analyte to the library of candidate affinity agents adhered to the array. Analytes such as those described herein above may be used. The analyte or analytes are in fluid phase or are contained within a fluid phase, and preferably are in an analyte solution. Some or all of the analyte or analytes may be coupled to a label.

The analyte may be exposed to the array in a number of different ways, some of which are known in the field. Such exposure techniques also include those discussed herein. For example, the analyte may be exposed to the array via convection, via incubation, via stagnant exposure, etc. The concentration of the analyte that is exposed to the array may be different and perhaps of increasing stringency in a series of experiments.

The analyte may be exposed to the array at the same time as an interfering substance to allow for competitive binding.

In certain implementations, titration of increasing concentration of analyte can be performed in order to generate binding curves. Here, analysis of the array would help to determine the concentration of the analyte at which binding is half maximal, which is usually equivalent to the dissociation constant.

The method further comprises analyzing the array to thereby identify the analyte-specific affinity agents. This analysis may comprise a single or multiple rounds, for example, as described herein above, and certain rounds may be duplicated more than once for reasons such as statistical significance.

Analysis of the array may comprise a single or multiple techniques and certain techniques may be duplicated more than once for reasons such as statistical significance. Any technique useful to analyze the array may be utilized such as, for example, use of an optical detector such as a fluorescence detector, use of electromagnetic radiation, use of algorithms to determine such conclusions as results of competitive binding studies, kinetic studies, etc.

In one implementation, the analysis of the array to identify analyte-specific affinity agents comprises measuring a signal, wherein analyte-specific affinity agents are identified if the signal is different than a background. This signal may be an optical signal (e.g., fluorescence), an electrical signal (e.g., current, voltage, resistance, capacitance, inductance, a combination thereof, or others known in the field), a thermal signal (e.g., radiation, thermal energy), a mechanical signal, etc.

For example, the analyte may be labeled with a quantum dot of certain characteristic excitation and emission wavelengths and an interfering substance may be labeled with a different quantum dot of different characteristic excitation and emission wavelengths. These analytes may be exposed to the array at the same time to allow for competitive binding. In this example, the affinity agent/s that bind's to the analyte and not to the interfering substance may be selected.

In another example, noncompetitive hybridizations can be performed for the analyte and the interferent. When the array is analyzed, those affinity agents possessing affinity towards the analyte, but not the interferent are selected.

In yet another embodiment, affinity agents can be found that tolerate variations in analyte subtype or structure, binding to several analytes by passing each analyte successively or in conjunction across the array. When the array is analyzed, those that bind specifically to all of the desired analytes are selected.

It may be useful in some applications to prescreen the array for affinity agents that bind either to the quantum dot or to the label (whatever that may be, for example, a fluorescein dye, another organic dye, etc). In this case, the analysis of the array may comprise a subtraction of the affinity agents that bind to the label such that the number of false positives may be reduced.

In certain implementations, the analysis of the array may comprise use of an algorithm. This may be helpful in, for example, determining certain properties of the affinity agents that are selected. For example, titrations of increasing concentration of analyte can be performed in order to generate binding curves. The concentration at which binding is half maximal is usually equivalent to the dissociation constant.

Use of an implementation that utilizes an array may, in certain instances, allow for pre-determination of certain characteristics of the affinity agent that would not be readily determined by selection of an affinity agent in solution. For example, because the affinity agents are attached to an array, the behavior of the affinity agent when involved in a surface or heterogeneous interaction may be determined. This behavior may be different than if the affinity agent were in solution (i.e., difference between heterogeneous and homogeneous reactions).

If the label is a fluorophor, the array may be analyzed using a fluorescence detector. Variations to this method, specific steps or parts of it are, of course, are possible.

In accordance with another aspect of the invention, a method is provided for characterizing analyte-specific affinity agents having affinity for an analyte. A presently preferred implementation of this method is outlined in FIG. 7.

The method comprises providing a library of candidate affinity agents wherein the library of candidate affinity agents comprises genomers. Examples of providing a library of candidate affinity agents are described herein.

The method also comprises providing an analyte, and exposing the analyte to the library of candidate affinity agents to cause selected ones of the candidate affinity agents to react with the analyte to form reaction products, where the reaction products are associated with certain analyte-specific affinity agents.

The method also comprises segregating the reaction products from the remainder of the library of candidate affinity agents. “Segregation” or “segregating” as used herein is used in its broad meaning. It includes any method that separates, divides, isolates, or otherwise segregates the reaction products from the remainder of the library of candidate affinity agents. For example, it may include physical and chemical separation. It may be facilitated by use of filters, such as nitrocellulose filters, chromatography, size exclusion principles, mass discrimination such as centrifugation, adsorption to a surface, etc. Other filtration techniques that are known in the field may be employed as well. The specific technique elected in a given application will likely depend on such factors as the application itself, the analyte, and the type of affinity agent sought. If, for example, the library of affinity agents comprises nucleic acids and the reaction products comprise analyte-specific affinity agents bound to the analyte, then the reaction products may be segregated from the remainder of the library via use of a nitrocellulose filter.

The method also comprises amplifying the analyte-specific affinity agents associated with the reaction products to form an enhanced library of affinity agents to thereby identify analyte-specific affinity agents. “Amplification” or “amplifying” as used herein is used in its broad meaning in the field. It includes any method that increases the concentration or copies of a particular molecule, ion, entity, or group thereof. For example, it may include use of enzymatic reactions such as polymerase chain reaction (PCR) and variations thereof, chemical reactions, primer-initiated amplification, random amplification, amplification without primers, ligation of fragments of digested genomic DNA, double-sided adapters, random hexomers used as “primers,” etc.

If the affinity agents to be amplified include genomers, the following types of strategies are examples of those that may be used to aid in amplification of genomers. First, these affinity agents may be amplified using methods known in the field to amplify the particular type of affinity agent. For instance, if the genomer comprises nucleic acids, then methods of amplifying, for example, genomic DNA may be used. Second, the genomers may be coupled to handles, which are molecules that aid in the amplification process. These handles may or may not include naturally occurring sequences, and preferably but optionally they do not interact with the analyte. For instance, endonucleases are used to digest genomic material at specific locations. Nucleic acid handles with a primer binding site in addition to a segment known to correspond with the digested sticky ends of the genomic material are ligated onto the genomers in the library. Following the amplification, in this example, when the analyte-specific affinity agent has been identified or otherwise characterized, these handles may be removed by another digestion.

Affinity agents discovered through the methods described herein sometimes may not be of sufficient affinity to be useful in the desired application. Accordingly, a chimeric analyte-specific affinity agent may be developed. A chimeric analyte-specific affinity agent may comprise at least two of the affinity agents characterized by methods described herein. Or, a chimeric analyte-specific affinity agent may comprise at least two molecules or entities where at least one is an analyte-specific affinity agent characterized by the methods described herein. The other molecule may be an analyte-specific affinity agent characterized by the methods described herein or it may be an affinity agent selected via other methods or it may be a different molecule or entity.

The two or more molecules or entities may be linked together to form an analyte-specific affinity agent of greater affinity or an analyte-specific affinity agent possessing combinations of the properties of the individual affinity agent components. In some embodiments, one of the molecules forming the chimeric affinity agent does not possess affinity for the analyte, but possesses some other function, such as enzymatic activity, which is modified upon linking it to an affinity agent.

Appropriate linkers may include, without limitation: polyethylene glycol, nucleic acids, peptides, carbon chains or other linkers known in the field. Linkers can sometimes be indirect such as attachment of affinity agents to a substrate in proximity of each other. In some embodiments, linkers are inert, exhibiting low or no affinity for the analyte.

In accordance with yet another aspect of the invention, an apparatus is provided for characterizing analyte-specific affinity agents having affinity for an analyte. The apparatus comprises an analyte receiver that receives the analyte, as described herein above.

It also comprises an array upon which a library of candidate affinity agents is attached. The array preferably comprises a microarray. The array is in fluid communication with the analyte receiver so that the analyte contacts the array when the analyte is received at the analyte receiver, which contact causes the analyte to contact the array to thereby expose the library of candidate affinity agents attached to the array to the analyte, and which contact causes selected ones of the candidate affinity agents to react with the analyte to form reaction products, and to create an output fluid that comprises the reaction products.

The apparatus also preferably comprises an analyzer that analyzes the output fluid to characterize the analyte-specific affinity agents associated with the respective reaction products.

In accordance with another aspect of the invention that is separate but related, an apparatus is provided that comprises an array upon which a library of candidate affinity agents comprising a genomer is attached. The array preferably comprises a microarray.

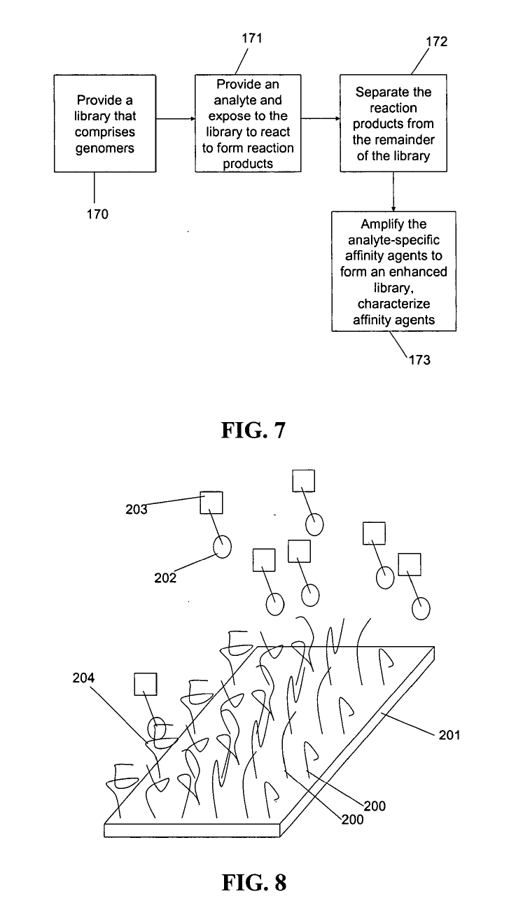

FIG. 8 shows a presently preferred embodiment of apparatus according to the aspect of the invention that can be utilized to identify affinity agents for a particular analyte or group of analytes. A library 200 is attached to an array 201. The analyte 202 is coupled to a label 203. When the analyte with the label is exposed to the library on the array, the analyte binds to affinity agents 204.

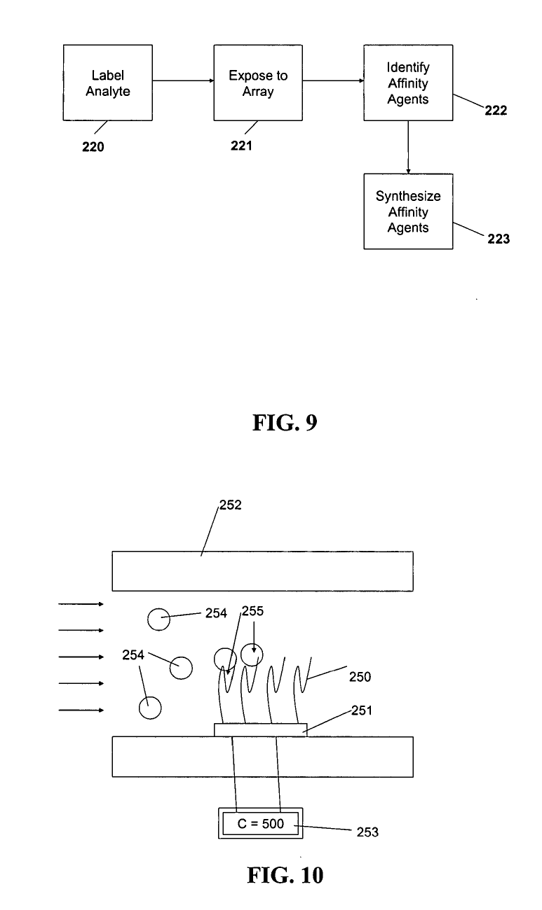

FIG. 9 is a block diagram of an illustrative implementation of a method that may be utilized to identify affinity agents. The analyte is labeled 220 and then it is exposed to the array 221. Affinity agents are identified 222 and these affinity agents are synthesized 223 for use in, for example, a sensor application.

As has been mentioned herein above, analyte-specific affinity agents that are identified or otherwise characterized according to each of the inventive methods described herein constitute additional aspects of the invention.

There are many applications in which the inventive methods and apparatus described herein may be employed. Some illustrative examples include identifying affinity agents for breath constituents, volatile organic compounds, oligosaccharides or glycoproteins, small molecules, etc.

Acetone is an example of a breath constituent that is a small molecule which is difficult to bind to a surface or to a fluorophore without significantly modifying its chemistry. The surface adsorption method provides a manner of characterizing affinity agents to small molecules of this nature.

In general, affinity agents can be used in a variety of settings including as extractants for purification, therapeutics or as a detection element or interactant on a number of different sensors including, without limitation, optical sensors, thermal sensors (e.g., thermoelectric using a thermopile or pyroelectric detection element), gravimetric sensors, electrochemical sensors, infrared sensors, etc. The sensors can be used in a variety of different environments such as, without limitation, breath, blood, water, fluid, gas, urine, spinal fluid, etc.

FIG. 10 shows an embodiment of a sensor according to the invention that utilizes affinity agents. An affinity agent 250 is immobilized on the surface of a sensor 251. The sensor is placed in an encasement 252 and is connected to an output 253. The analyte 254 comes in contact with the affinity agent 250 and forms reaction products 255. The sensor 251 detects the reaction and reports the concentration or presence of the analyte 254 on the output 253. As mentioned previously herein above, various types of sensors may be used with the analyte-specific affinity agents selected using the methods described herein, including thermoelectric sensors.

In accordance with yet another aspect of the invention, a kit is provided for characterizing analyte-specific affinity agents having affinity for an analyte, the kit comprises an analyte receiver that receives the analyte. The analyte may take any of the forms described herein above.

The kit further comprises a surface upon which a library of candidate affinity agents is adhered. The surface preferably is matable with the analyte receiver so that, when such contact is made, the analyte contacts the surface when the analyte is received at the analyte receiver. The contact causes the analyte to contact the surface to thereby expose the library of candidate affinity agents adhered to the surface to the analyte, and the contact causes selected ones of the candidate affinity agents to react with the analyte to form reaction products, and to create an output fluid that comprises the reaction products.

The kit may further comprise an analyzer for analyzing the output fluid to characterize the analyte-specific affinity agents associated with the respective reaction products.

In accordance with another aspect of the invention, a kit is provided for characterizing analyte-specific affinity agents having affinity for an analyte, wherein the kit comprises a library of candidate affinity agents adhered to an array, and preferably a microarray.

In each of these kits, the library of candidate affinity agents may comprise a genomer, such as the genomers described herein above.

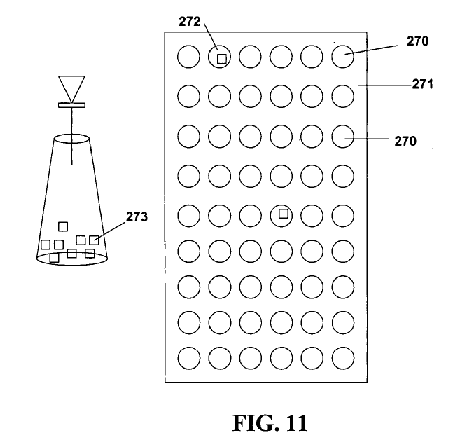

FIG. 11 shows an embodiment of a kit for characterizing analyte-specific affinity agents along with a bottle containing the analyte of interest 273. The kit comprises genomers 270 attached to a microarray 271. This kit may be used to identify specific genomers 272 that react with an analyte of interest 273.

Additional advantages and modifications will readily occur to those skilled in the art. For example, genomers may comprise polymers or conjugates of biomolecules. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

We claim:

1. A method for characterizing analyte-specific affinity agents having affinity for an analyte, the method comprising:
providing a library of candidate affinity agents;
providing a surface suitable for adhesion of the library of candidate affinity agents, and exposing the library of candidate affinity agents to the surface to thereby adhere the library of candidate affinity agents to the surface;
providing an analyte, and causing the analyte to contact the surface to thereby expose the library of candidate affinity agents adhered to the surface to the analyte, to cause selected ones of the candidate affinity agents to react with the analyte to form reaction products, and to create an output fluid that comprises the reaction products;
analyzing the output fluid to characterize the analyte-specific affinity agents associated with the respective reaction products.

2. A method as recited in claim 1, wherein the provision of the library of candidate affinity agents comprises providing non-naturally occurring nucleic acids within the library of candidate affinity agents.

3. A method as recited in claim 1, wherein the provision of the library of candidate affinity agents comprises providing a peptide within the library of candidate affinity agents.

4. A method as recited in claim 1, wherein the provision of the library of candidate affinity agents comprises providing a genomer within the library of candidate affinity agents.

5. A method as recited in claim 4, wherein the genomer comprises at least a portion of a human genome.

6. A method as recited in claim 5, wherein the human genome comprises a coding portion of the human genome.

7. A method as recited in claim 1, wherein provision of the surface comprises providing the surface to comprise a silica.

8. A method as recited in claim 1, wherein the provision of the surface comprises providing the surface to comprise a spin column.

9. A method as recited in claim 1, wherein the exposure of the library of candidate affinity agents to the surface comprises adsorbing the library of candidate affinity agents to the surface nonspecifically.

10. A method as recited in claim 1, wherein the provision of the analyte comprises providing the analyte to include at least one of proteins, lipids, carbohydrates, and toxins.

11. A method as recited in claim 1, wherein the provision of the analyte comprises providing the analyte to include acetone.

12. A method as recited in claim 1, wherein the provision of the analyte comprises providing the analyte to include a small molecule.

13. A method as recited in claim 1, wherein the provision of the analyte comprise providing the analyte to include at least one constituent of human breath.

14. A method as recited in claim 1, wherein the analysis of the output fluid comprises using differential binding.

15. A method as recited in claim 1, wherein the analysis of the output fluid comprises isolating the analyte-specific affinity agents.

16. A method as recited in claim 1, wherein the analysis of the output fluid comprises identifying the analyte-specific affinity agents.

17. An apparatus for characterizing analyte-specific affinity agents having affinity for an analyte, the apparatus comprising:
an analyte receiver that receives the analyte;
a surface upon which a library of candidate affinity agents is adhered, the surface being in fluid communication with the analyte receiver so that the analyte contacts the surface when the analyte is received at the analyte receiver, which contact causes the analyte to contact the surface to thereby expose the library of candidate affinity agents adhered to the surface to the analyte, and which contact causes selected ones of the candidate affinity agents to react with the analyte to form reaction products, and to create an output fluid that comprises the reaction products; and
an analyzer that analyzes the output fluid to characterize the analyte-specific affinity agents associated with the respective reaction products.

18. A method for characterizing analyte-specific affinity agents having affinity for an analyte, the method comprising:
providing a library of candidate affinity agents wherein the library of candidate affinity agents comprises genomers; and,
providing an analyte, and exposing the analyte to the library of candidate affinity agents to thereby characterize the analyte-specific affinity agents.

19. A method as recited in claim 18, wherein the provision of the library of candidate affinity agents consists essentially of genomers.

20. A method as recited in claim 18, wherein the provision of the library of candidate affinity agents consists essentially of genomers wherein the genomers comprise at least a portion of a human genome.

21. A method as recited in claim 20, wherein the genomers comprise a non-coding portion of the human genome.

22. A method as recited in claim 20, wherein the genomers comprise a coding portion of the human genome.

23. A method as recited in claim 18, wherein the provision of the analyte comprises providing the analyte to comprise at least one of proteins, lipids, carbohydrates and toxins.

24. A method as recited in claim 18, wherein the provision of the analyte comprises providing the analyte to comprise acetone.

25. A method as recited in claim 18, wherein the provision of the analyte comprises providing the analyte to comprise a small molecule.

26. A method as recited in claim 18, wherein the provision of the analyte comprise providing the analyte to comprise at least one constituent of human breath.

27. An apparatus for characterizing analyte-specific affinity agents having affinity for an analyte, the apparatus comprising:
an analyte receiver that receives the analyte;
a surface upon which a library of candidate affinity agents comprising genomers is adhered, the surface being in fluid communication with the analyte receiver so that the analyte contacts the surface when the analyte is received at the analyte receiver, which contact causes the analyte to contact the surface to thereby expose the library of candidate affinity agents adhered to the surface to the analyte, and which contact causes selected ones of the candidate affinity agents to react with the analyte to form reaction products, and to create an output fluid that comprises the reaction products; and
an analyzer that analyzes the output fluid to characterize the analyte-specific affinity agents associated with the respective reaction products.

28. A method for characterizing analyte-specific affinity agents having affinity for an analyte, the method comprising:
providing a library of candidate affinity agents wherein the library of candidate affinity agents comprises genomers, and further wherein the library of candidate affinity agents is attached to an array;
providing an analyte, and exposing the analyte to the library of candidate affinity agents attached to the array;
analyzing the array to thereby characterize the analyte-specific affinity agents.

29. A method as recited in claim 28, wherein the provision of the library of candidate affinity agents comprises providing the library of candidate affinity agents attached to a microarray.

30. A method as recited in claim 28, wherein the analysis of the array to characterize analyte-specific affinity agents further comprises measuring a signal, wherein analyte-specific affinity agents are characterized if the signal is different than a background.

31. An apparatus for characterizing analyte-specific affinity agents having affinity for an analyte, the apparatus comprising an array upon which a library of candidate affinity agents comprising a genomer is attached.

32. An apparatus as recited in claim 31, wherein the array comprises a microarray.

33. A method for characterizing analyte-specific affinity agents having affinity for an analyte, the method comprising:
providing a library of candidate affinity agents wherein the library of candidate affinity agents comprises genomers;
providing an analyte, and exposing the analyte to the library of candidate affinity agents to thereby cause selected ones of the candidate affinity agents to react with the analyte to form reaction products, where the reaction products are associated with certain analyte-specific affinity agents;
segregating the reaction products from the remainder of the library of candidate affinity agents; and
amplifying the analyte-specific affinity agents associated with the reaction products to form an enhanced library of affinity agents to thereby characterize analyte-specific affinity agents.

34. An analyte-specific affinity agent having affinity for an analyte that is characterized according to the method of claim 1.

35. An analyte-specific affinity agent having affinity for an analyte that is characterized according to the method of claim 18.

36. An analyte-specific affinity agent having affinity for an analyte that is characterized according to the method of claim 28.

37. An analyte-specific affinity agent having affinity for an analyte that is characterized according to the method of claim 33.

38. A chimeric analyte-specific affinity agent having affinity for an analyte, comprising at least two molecules, wherein at least one of the two molecules is an analyte-specific affinity agent characterized according to at least one of the methods of the method of claim 1, the method of claim 18, the method of claim 28, and the method of claim 33.

39. A kit for characterizing analyte-specific affinity agents having affinity for an analyte, the kit comprising:
an analyte receiver that receives the analyte;
a surface upon which a library of candidate affinity agents is adhered, the surface being matable with the analyte receiver so that the analyte contacts the surface when the analyte is received at the analyte receiver, which contact causes the analyte to contact the surface to thereby expose the library of candidate affinity agents adhered to the surface to the analyte, and which contact causes selected ones of the candidate affinity agents to react with the analyte to form reaction products, and to create an output fluid that comprises the reaction products; and
an analyzer for analyzing the output fluid to characterize the analyte-specific affinity agents associated with the respective reaction products.
